(19) United States
(12) Patent Application Publication
(10) Pub. No.: US 2008/0085513 A1
(43) Pub. Date: Apr. 10, 2008

(54) In situ nucleic acid array synthesis compositions

(76) Inventors: Eric Leproust, San Jose, CA (US); Bill J. Peck, Mountain View, CA (US); Xiaohua C. Huang, Mountain View, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/545,931

(22) Filed: Oct. 10, 2006

(51) Int. Cl. C12Q 1/68; C12M 1/34

(52) U.S. Cl. 435/6; 435/287.2; 427/2.11

(57) ABSTRACT

In situ nucleic acid synthesis compositions are provided. The compositions include a solvent; and at least one of: a viscosity modifier; and a surface tension modifier. During use, the compositions further include one of a phosphoramidite and an activator. Also provide are methods of using the compositions, e.g., in the synthesis of nucleic acid arrays, and systems kits for practicing the methods.

### BACKGROUND

Nucleic acid arrays (such as DNA or RNA arrays), are known and are used, for example, as diagnostic or screening tools. Such arrays include regions of usually different sequence polynucleotides arranged in a predetermined configuration on a surface of a solid support, such as a glass slide. These regions (sometimes referred to as “features”) are positioned at respective locations (“addresses”) on the surface of the solid support. The arrays, when exposed to a sample, will exhibit an observed binding pattern depending the probe molecules in the feature location and the target molecules present in the sample. This binding pattern can be detected upon interrogating the array, where the interrogation protocol employed depends on the labeling scheme that is used.

For example, in a nucleic acid array assay, all polynucleotide targets (for example, DNA) in a sample can be labeled with a fluorescent label, and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide targets of the sample.

Nucleic acid arrays can be fabricated by depositing previously obtained biopolymers onto a substrate, or by in situ synthesis methods. The in situ fabrication methods include those described in U.S. Pat. No. 6,180,351 and WO 98/41531, and the references cited therein.

### SUMMARY

In situ nucleic acid synthesis compositions are provided. The compositions include a solvent; and at least one of: a viscosity modifier; and a surface tension modifier. During use, the compositions further include one of a nucleic acid precursor, e.g., nucleic acid monomeric precursor thereof, e.g., phosphoramidite, and an activator. Also provide are methods of using the compositions, e.g., in the synthesis of nucleic acid arrays, and systems kits for practicing the methods.

### BRIEF DESCRIPTION OF THE FIGURES

FIG. 1 illustrates a substrate carrying multiple arrays, such as may be fabricated by methods of the present invention.

FIG. 2 is an enlarged view of a portion of FIG. 1 showing multiple ideal spots or features.

FIG. 3 is an enlarged illustration of a portion of the substrate in FIG. 2.

FIG. 4 schematically illustrates an array fabrication system according to an embodiment of the present invention.

### DEFINITIONS

The term “monomer” as used herein refers to a chemical entity that can be covalently linked to one or more other such entities to form a polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). In the art synthesis of nucleic acids of this type utilizes an initial substrate-bound monomer that is generally used as a building-block in a multi-step synthesis procedure to form a complete nucleic acid. A “biomonomer” references a single unit, which can be linked with the same or other biomonomers to form a biopolymer (e.g., a single amino acid or nucleotide with two linking groups, one or both of which may have removable protecting groups).

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “amino acid” is intended to include not only the L, D- and nonchiral forms of naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), but also modified amino acids, amino acid analogs, and other chemical compounds which can be incorporated in conventional oligopeptide synthesis, e.g., 4-nitrophenylalanine, isoglutamic acid, isoglutamine, ε-nicotinoyl-lysine, isonipecotic acid, tetrahydroisoquinoleic acid, α-aminoisobutyric acid, sarcosine, citrulline, cysteic acid, t-butylglycine, t-butylalanine, phenylglycine, cyclohexylalanine, β-alanine, 4-aminobutyric acid, and the like.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure. In the practice of the instant invention, oligomers will generally comprise about 2-50 monomers, preferably about 2-20, more preferably about 3-10 monomers.

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

A “biopolymer” is a polymer of one or more types of repeating units. Biopolymers are typically found in biological systems (although they may be made synthetically) and may include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. For example, a “biopolymer” may include DNA (including cDNA), RNA, oligonucleotides, and PNA and other polynucleotides as described in U.S. Pat. No. 5,948,902 and references cited therein (all of which are incorporated herein by reference), regardless of the source.

The term “biomolecule” means any organic or biochemical molecule, group or species of interest that may be formed in an array on a substrate surface. Exemplary biomolecules include peptides, proteins, amino acids and nucleic acids. The term “ligand” as used herein refers to a moiety that is capable of covalently or otherwise chemically binding a compound of interest. The arrays of solid-supported ligands produced by the methods can be used in screening or separation processes, or the like, to bind a component of interest in a sample. The term “ligand” in the context of the invention may or may not be an “oligomer” as defined above. However, the term “ligand” as used herein may also refer to a compound that is “pre-synthesized” or obtained commercially, and then attached to the substrate. The term “sample” as used herein relates to a material or mixture of materials, typically, although not necessarily, in fluid form, containing one or more components of interest.

A biomonomer fluid or biopolymer fluid reference a liquid containing either a biomonomer or biopolymer, respectively (typically in solution). The term “peptide” as used herein refers to any polymer compound produced by amide formation between an α-carboxyl group of one amino acid and an α-amino group of another group.

The term “oligopeptide” as used herein refers to peptides with fewer than about 10 to 20 residues, i.e., amino acid monomeric units.

The term “polypeptide” as used herein refers to peptides with more than 10 to residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single-stranded nucleotide multimers of from about 10 up to about 200 nucleotides in length, e.g., from about 25 to about 200 nt, including from about 50 to about 175 nt, e.g. 150 nt in length The term “polynucleotide” as used herein refers to single- or double-stranded polymers composed of nucleotide monomers of generally greater than about 100 nucleotides in length.

An “array,” or “chemical array” used interchangeably includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. As such, an addressable array includes any one or two or even three-dimensional arrangement of discrete regions (or “features”) bearing particular biopolymer moieties (for example, different polynucleotide sequences) associated with that region and positioned at particular predetermined locations on the substrate (each such location being an “address”). These regions may or may not be separated by intervening spaces. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc.

In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays may be fabricated using drop deposition from pulse jets of either precursor units (such as nucleotide or amino acid monomers) in the case of in situ fabrication, or the previously obtained biomolecule, e.g., polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein.

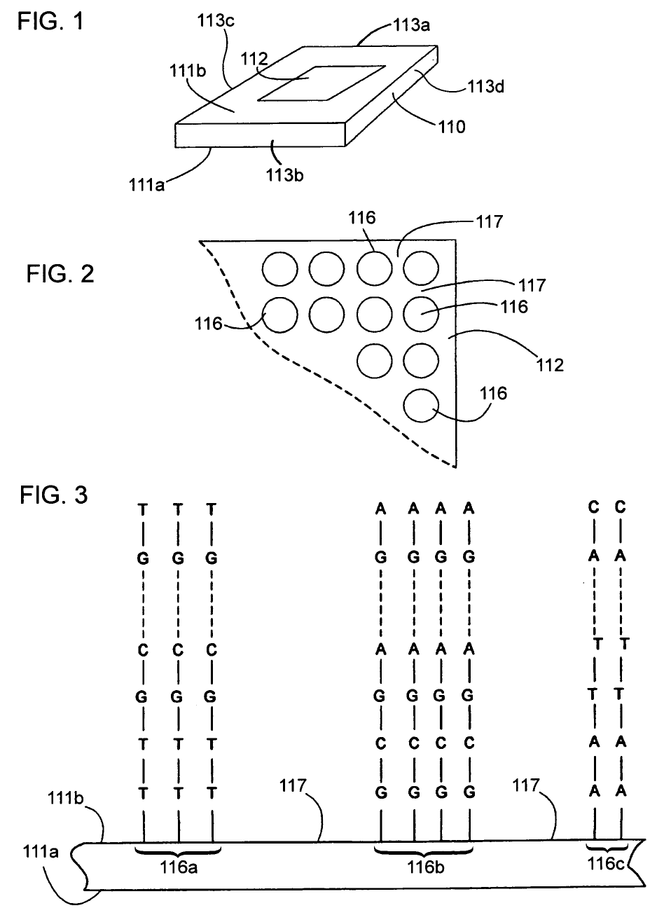

An exemplary chemical array is shown in FIGS. 1-3, where the array shown in this representative embodiment includes a contiguous planar substrate 110 carrying an array 112 disposed on a surface 111b of substrate 110. It will be appreciated though, that more than one array (any of which are the same or different) may be present on surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the surface 111b, with regions of the rear surface 111b adjacent the opposed sides 113c, 113d and leading end 113a and trailing end 113b of slide 110, not being covered by any array 112. A second surface 111a of the slide 110 does not carry any arrays 112.

Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymer ligands, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

The substrate may be porous or non-porous. The substrate may have a planar or non-planar surface.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

An array “assembly” includes a substrate and at least one chemical array, e.g., on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

“Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. “Stably attached” or “stably associated with” means an item's position remains substantially constant where in certain embodiments it may mean that an item's position remains substantially constant and known.

A “web” references a long continuous piece of substrate material having a length greater than a width. For example, the web length to width ratio may be at least 5/1, 10/1, 50/1, 100/1, 200/1, or 500/1, or even at least 1000/1.

“Flexible” with reference to a substrate or substrate web, references that the substrate can be bent 180 degrees around a roller of less than 1.25 cm in radius.

The substrate can be so bent and straightened repeatedly in either direction at least 100 times without failure (for example, cracking) or plastic deformation. This bending must be within the elastic limits of the material. The foregoing test for flexibility is performed at a temperature of 20° C.

“Rigid” refers to a material or structure which is not flexible, and is constructed such that a segment about 2.5 by 7.5 cm retains its shape and cannot be bent along any direction more than 60 degrees (and often not more than 40, 20, 10, or 5 degrees) without breaking.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

“Contacting” means to bring or put together. As such, a first item is contacted with a second item when the two items are brought or put together, e.g., by touching them to each other.

“Depositing” means to position, place an item at a location-or otherwise cause an item to be so positioned or placed at a location. Depositing includes contacting one item with another. Depositing may be manual or automatic, e.g., “depositing” an item at a location may be accomplished by automated robotic devices.

By “remote location,” it is meant a location other than the location at which the array (or referenced item) is present and hybridization occurs (in the case of hybridization reactions). For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information means transmitting the data representing that information as signals (e.g., electrical, optical, radio signals, and the like) over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

An array “package” may be the array plus only a substrate on which the array is deposited, although the package may include other features (such as a housing with a chamber).

A “chamber” references an enclosed volume (although a chamber may be accessible through one or more ports). It will also be appreciated that throughout the present application, that words such as “top,” “upper,” and “lower” are used in a relative sense only.

It will also be appreciated that throughout the present application, that words such as “cover”, “base” “front”, “back”, “top”, are used in a relative sense only. The word “above” used to describe the substrate and/or flow cell is meant with respect to the horizontal plane of the environment, e.g., the room, in which the substrate and/or flow cell is present, e.g., the ground or floor of such a room.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

A “pulse jet” is any device which can dispense drops of a fluid composition in the formation of an array. Pulse jets operate by delivering a pulse of pressure (such as by a piezoelectric or thermoelectric element) to liquid adjacent an outlet or orifice such that a drop will be dispensed therefrom.

A “fluid composition” or “fluid formulation” is a composition or formulation that includes a high boiling point solvent, a probe precursor dissolved in the solvent, a viscosity modifier and/or a surface tension modifier, and a nucleic acid precursor, e.g., phosphoramidite or another reagent, such as an activator, which composition is suitable for use in a pulse jet.

A “group” in relation to a chemical formula, includes both substituted and unsubstituted forms of the group where any substituents do not interfere with the desired reactions.

A “phospho” group includes a phosphodiester, phosphotriester, and H-phosphonate groups as defined in connection with formula (I) above, while a “phosphite” includes a phosphoramidite.

Nucleic acid arrays can be fabricated by depositing previously obtained nucleic acids onto a substrate, or by in situ synthesis methods. The in situ fabrication methods include those described in U.S. Pat. No. 5,449,754 for synthesizing peptide arrays, and in U.S. Pat. No. 6,180,351 and WO 98/41531 and the references cited therein for synthesizing polynucleotide arrays. The in situ method for fabricating a polynucleotide array typically follows, at each of the multiple different addresses at which features are to be formed, the same conventional iterative sequence used in forming polynucleotides on a support. Typically these methods use a nucleoside reagent of the formula:

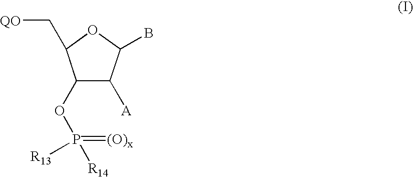

in which:

A represents H, alkyl, or another substituent which does not interfere in the coupling of compounds of formula (I) to form polynucleotides according to the in situ fabrication process;

B is a purine or pyrimidine base whose exocyclic amine functional group is optionally protected;

Q is a conventional protective group for the 5′—OH functional group;

x=0 or 1 provided:

a) when x=1:

R₁₃ represents H and R₁₄ represents a negatively charged oxygen atom; or

R₁₃ is an oxygen atom and R₁₄ represents either an oxygen atom or an oxygen atom carrying a protecting group; and

b) when x=0, R₁₃ is an oxygen atom carrying a protecting group and R₁₄ is either a hydrogen or a di-substituted amine group.

When x is equal to 1, R₁₃ is an oxygen atom and R₁₄ is an oxygen atom, the method is in this case the so-called phosphodiester method; when R₁₄ is an oxygen atom carrying a protecting group, the method is in this case the so-called phosphotriester method.

When x is equal to 1, R₁₃ is a hydrogen atom and R₁₄ is a negatively charged oxygen atom, the method is known as the H-phosphonate method.

When x is equal to 0, R₁₃ is an oxygen atom carrying a protecting group and R₁₄ is either a halogen, the method is known as the phosphite method and; when x=0, R₁₃ is an oxygen atom carrying a protecting group, and R₁₄ is a leaving group of the disubstituted amine type, the method is known as the phosphoramidite method.

The conventional sequence used to prepare an nucleic acid, e.g., oligonucleotided using reagents of the type of formula (I), basically follows the following steps: (a) coupling a selected nucleoside reagent, (e.g., phosphoramidite) through a phosphite linkage to a functionalized support in the first iteration, or a nucleoside bound to the substrate (i.e. the nucleoside-modified substrate) in subsequent iterations; (b) optionally blocking (“capping”) unreacted hydroxyl groups on the substrate bound nucleoside; (c) oxidizing the phosphite linkage of step (a) to form a phosphate linkage; and (d) removing the protecting group (“deprotection”) from the now substrate bound nucleoside coupled in step (a), to generate a reactive site for the next cycle in which these steps are repeated. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in a known manner. The nucleoside reagent in (a) generally requires activation by a suitable activator such as tetrazole.

The foregoing methods of preparing polynucleotides are described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643, EP 0294196, and elsewhere. The phosphoramidite and phosphite triester approaches are most broadly used, but other approaches which may be employed include, but are not limited to, the phosphodiester approach, the phosphotriester approach and the H-phosphonate approach.

In the case of array fabrication, different monomers may be deposited at different addresses on the substrate during any one iteration so that the different features of the completed array will have different desired biopolymer sequences. One or more intermediate further steps may be required in each iteration, such as the conventional oxidation and washing steps. One particularly useful way of depositing monomers is by depositing drops each containing a monomer from a pulse jet spaced apart from the substrate surface, onto the substrate surface. Such techniques are described in detail in, for example, U.S. Pat. No. 6,242,266, U.S. Pat. No. 6,232,072, U.S. Pat. No. 6,180,351, and U.S. Pat. No. 6,171,797. Prior art pulse jets are available commercially for use in ink printing which are provided with an indicated viscosity or viscosity range at which the pulse jet will function best.

“Lower alkyl group” or other “lower” group references either such group with from 1 to 6 C atoms (such as 2, 3, 4, or 5 C atoms).

A “blocked” hydroxy group or amino group references a hydroxy group (—OH) or an amino group (R₂NH— or —NH₂, where R represents a substituent on the amino group) in which any free H has been replaced by a protecting group which renders the hydroxy or amino unreactive under the conditions of an in situ biopolymer fabrication process in which it is used.

A “blocked polyhydric polymer” is any polymer in which the polymer molecules each has multiple blocked hydroxy groups. An example of a blocked polyhydric polymer is a blocked polyalkylene glycol which can be thought of as a polymer of alkylene glycol units (such as a lower alkylene glycol, for example ethylene glycol).

“Unblocked-hydroxy free” and unblocked-amino free” polymers refer to polymers which do not have any unblocked hydroxy or amino groups (that is, do not have an —OH or an —NR¹H group, where R¹ may be H or another substituent).

By “same concentration” of different solutions is referenced that the concentrations, on a molar basis, are within 20% of one another (although they may be within 15, 10, 5 or 2% of one another).

A “molecular weight” of a polymer which may be a mixture of polymers, is taken as the average molecular weight of the mixture. Molar concentrations of such mixtures are based on the average molecular weight.

A “high molecular weight polyethylene glycol” has a molecular weight higher than about 35 kDa to about 1 MDa and above, for example 5 MDa. A “low molecular weight polyethylene glycol” has a molecular weight lower than about 35 kDa.

“Fluid” is used herein to mean a liquid.

A “high boiling point” refers to a boiling of higher than about 150° C.

“Coupling yield” refers to the percent yield of any coupling reaction between a nucleoside phosphoramidite and a free hydroxyl group in the presence of an activator.

All viscosities herein are in centipoise (“cp”) at 25° C. unless otherwise noted and are based on a rotational rheometer at a shear rate of 400 sec⁻¹.

### DETAILED DESCRIPTION

In situ nucleic acid synthesis compositions are provided. The compositions include a solvent; and at least one of: a viscosity modifier; and a surface tension modifier. During use, the compositions further include one of a nucleic acid precursor, e.g., phosphoramidite, and an activator. Also provide are methods of using the compositions, e.g., in the synthesis of nucleic acid arrays, and systems kits for practicing the methods.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention.

Further, the dates of publication provided may be different from the actual publication dates which may need to be independently confirmed. It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, aspects of the invention include reagent compositions, methods of using the same in nucleic acid synthesis, in situ nucleic acid synthesis systems that include the subject reagent compositions, and kits that find use in practicing embodiments of the methods. Each of these aspects is now reviewed separately in greater detail.

### Compositions

The compositions of the invention are polar, aprotic composition that are compatible with in situ nucleic acid array synthesis protocols in which nucleic acids are produced on a surface of a support using a protocol in which reagent compositions of nucleic acid precursors (e.g., phosphoramidites or activators are sequentially deposited on the same location of the surface using a pulse jet device in an interative fashion to fabricate nucleic acids of a desired sequence and length at the surface location.

Because the compositions are compatible with in situ nucleic acid synthesis reactions, and particularly the coupling reactions of phosphoramide/nucleic acid prescursor reagents (i.e., the chemical reaction that occurs at the surface/liquid interface), the compositions are polor and aprotic, and have a relatively high dielectric constant. By relatively high dielectric constant is meant a dielectric constant that is about 30 or higher, such as about 43 or higher, where the dielectric constant may range in certain embodiments from about 43 to about 200, such as from about 60 to about 82.

The compositions are further characterized in that they are compatible with the jetting (i.e., ejection from a pulse-jet of a printhead) and printing (i.e., impact of the droplet on the solid support surface) aspects of in situ nucleic acid synthesis protocols. As such, the compositions have a viscosity, surface tension (both dynamic and static), contact angle, with both air and the surfaces that are contacted by the composition during use, and boiling point which are selected (based on proper selection of the components of the compositions), to be compatible with the jetting and printing parameters of in situ nucleic acid synthesis protocols.

Embodiments of the compositions have a viscosity that ranges from about 3 to about 30, such as from about 4 to about 15 and including from about 6 to about 8 cps. Embodiments of the compositions have a surface tension that ranges from about 25 to about 50 dyne/cm, such as from about 30 to about 45 dyne/cm and including from about 35 to about 40 dyne/cm. Embodiments of the compositions have a contact angle (with planar glass) which ranges from about 30 to about 90, such as from about 50 to about 80 and including from about 60 to about 700, as determined using the contact angle measurement assay described in _P.G. de Gennes “Wetting: statics and dynamics” Reviews of Modern Physics, 57, 3 (part 1), July 1985, p. 827-863. DOI: 10.1103/RevModPhys.57.827 (which can be obtained at the website having a address made up by place “http://” before “prola.aps.org/abstract/RMP/v57/i3/p827_{—}1”). Embodiments of the compositions have a boiling point that is about 80° C. or higher, such as about 100° C. or higher, and in certain embodiments ranges from about 80° C. to about 500° C. or higher, such as from about 100° C. to about 300° C. and including from about 150° C. to about 300° C.

In certain embodiments, sets of reagent compositions of different reagents are provided in which the physical properties of each reagent composition of the set are substantially the same, if not identical. For example, sets of 2 or more different reagent compositions (where each of the different reagent compositions of the set differs from each other by particular reagent, and in certain embodiments only by the reagent such that the remainder of the composition, e.g., the solvent vehicle, is identical), such as 3 or more, 4 or more, 5 or more, etc., are provided, where the physical properties of each reagent composition of the set are substantially the same, if not identical. An example of a set of reagent compositions is a set of nucleic acid synthesis reagent compositions made up of an A composition, a C composition, a T composition, a G composition, and a Tet composition, as reported in the experimental section, below. For such a set of compositions, despite the different phosphoramidite reagents present in each composition, the viscosity between each composition may vary about 1.5 cps or less, such as by about 1.0 cps or less, including by about 0.75 cps or less, e.g., 0.5 cps or less, 0.25 cps or less or even less.

As indicated above, the components of the compositions of the invention are selected and chosen, both in terms of identity and amount, to provide for the desired physical and nucleic acid synthesis reaction compatibility characteristics reviewed above. Aspects of the invention include compositions that include a solvent and at least one of a viscosity modifier and a surface tension modifier.

In certain embodiments, the solvents of the invention are polar, aprotic solvents that have a high boiling point, where solvents of interest include those that have a boiling point that is about 80° C. or higher, such as about 100° C. or higher, and in certain embodiments ranges from about 80° C. to about 500° C. or higher, such as from about 100° C. to about 300° C. and including from about 150° C. to about 300° C. Specific polar, aprotic solvents of interest may include, but are not limited to: alkylene carbonates, such as propylene carbonate (boiling point 240° C.); adiponitrile (boiling point 295° C.); dimethyl sulfoxide (DMSO) (boiling point 189° C.); sulfolane (boiling point 285° C.); and N-methyl acetamide (boiling point 205° C.); where the reported boiling points are as determined at STP. In certain embodiments, the composition does not include acetonitrile.

As indicated above, the compositions also include at least one of a viscosity modifier and a surface tension modifier. Where a viscosity modifier is present in the compositions, the viscosity modifier may be a viscosity enhancer or a viscosity reducer. Any viscosity modifier should be compatible with the chemistry in which it is to be used so that it does not adversely affect coupling yield as (defined above) by more than 5%. The viscosity modifier will also be soluble in the liquid containing the probe precursor. A viscosity enhancing polymer may be an unblocked-hydroxy free and unblocked-amino free polymer, or a blocked polyhydric polymer.

In certain embodiments, the viscosity modifier is made up of one or more polyalkylene glycols, e.g., having a C2 to C6 alkylene unit, such as polyethylene glycols, polypropylene glycols, etc. In certain embodiments, the viscosity modifier includes a polyethylene glycol (PEG) having a molecular weight of about 1 kDa or higher, such as about 20 kDa or higher, where the molecular weight of the PEG(s) in certain embodiments ranges from about 1 kDa to about 5 MDa, such as from about 30 to about 100 kDa.

In certain embodiments, the viscosity modifier is made up of a combination of two or more different viscosity modifiers, such as two or more different polyalkylene glycols, e.g., two or more different polyethylene glycols. In certain of these embodiments, the viscosity modifier includes at least one low molecular weight polyalkylene glycol and a least one high molecular weight polyalkylene glycol. Low molecular weight polyalkylene glycols are those having molecular weights ranging from about 250 Da to about 10 kDa, such as from about 500 Da to about 5 kDa and including from about 750 Da to about 2 kDa. High molecular weight polyalkylene glycols are those having a molecular weights ranging from about 10 kDa to about 10 MDa, such as from about 20 kDa to about 1 MDa and including from about 30 kDa to about 100 kDa. The amount of low molecular weight polyalkylene glycol present in the viscosity modifier may be chosen to impart to the composition a desired Newtonian impact on viscosity. The amount of high molecule weight polyalkylene glycol present in the viscosity modifier may be chosen to impart to the composition a desired non-Newtonian impact on viscosity. In certain embodiments, the amount ratio of low to high molecular weight polyalkylene glycols in the viscosity modifier ranges from about 0.1 to about 10,000, such as from about 1 to about 1,000 w/w.

In certain embodiments, the polyalkylene glycol(s) of the viscosity modifier is a blocked polyalkylene glycol, such as one having the formula II below:

B¹—O-(Alk-O)ₙ-Alk-O—B² (II)

where B¹ and B² are blocking groups (which may be the same or different), n is an integer (such as 2 to 10000, for example or 100 to 6000 or 200 to 3000), and Alk is an alkylene group (such as a lower alkylene group, for example ethylene (—CH₂—CH₂—)). Note that in formula (II) both terminal hydroxy groups are blocked by blocking groups B¹ or B² although some small number of the molecules may not have both hydroxy groups blocked provided any resulting small reduction in coupled product yield can be tolerated.

Suitable blocking groups for any hydroxy or amine may be any group that does not allow the blocked O or N to react with the probe precursor (or precursors where more than one is present) in the same liquid under the conditions used (for example, in the presence of tetrazole activator) to link one probe precursor to another on a substrate surface, or in any other manner does not adversely affect the yield of coupled product resulting from using that liquid in a cycle of the in situ probe synthesis. By “not adversely affecting” yield is referenced that any reduction in yield is less than 5%, 4%, 3%, 2%, 1% or 0.5% or even less than 0.2%. The foregoing percentages are based on a theoretical maximum of 100% such that if the yield without a compound of formula (II) was 98%, then the yield would not be less than 93% in the extreme case where yield is reduced by 5%.

Suitable blocking groups for use in typical in situ probe synthesis methods, such as phosphoramidite chemistry, include, but are not limited to: hydrocarbyl radicals, such as alkyl (for example lower alkyl groups) or aryl (for example, benzyl); or ester groups (for example, —C(O)OR or —OC(O)R, where R is an alkyl or aryl group such as a lower alkyl group); or ether groups (for example, —C—O—R where R is an alkyl or aryl group such as a lower alkyl group). Other blocking groups which may be used include any of those protecting groups used to protect the 3′ or 5′ hydroxy of a nucleoside phosphoramidite during linking to the hydroxy of a previously deposited nucleoside phosphoramidite or on the surface. Such blocking groups may be protecting groups, such as those described in “Protective groups in organic synthesis” by Theodora W. Greene and Peter G. M. Wuts, Wiley-interscience ISBN 0-471-62301-6 p. 68-117, and may be made by methods described therein or otherwise.

The amount of viscosity modifier, when present, in the compositions of the invention may vary. In certain embodiments, the viscosity modifier is present in the composition at a concentration ranging from about 10⁻⁶ g/mL to about 10⁻¹ g/mL, such as from about 10⁻⁴ g/mL to about 10⁻² g/mL and including from about 10⁻³ g/mL to about 10⁻² g/mL.

As indicated above, instead of or in addition to, the viscosity modifier, the compositions may include a surface tension modifier. Various surface tension modifiers may be used in the fluid compositions. The surface tension modifier may be any surfactant without a significant number of hydroxyl or amino functional groups, such that where surfactants include such groups, the concentration of such groups is about 10% or less, such as about 1% or less of the concentration of the active reagents.

Surfactants of interest include, but are not limited to: Alkyl poly(ethylene oxide), Alkyl polyglucosides, including Octyl glucoside and Decyl maltoside, Fatty alcohols including Cetyl alcohol and Oleyl alcohol, Cocamide MEA, cocamide DEA, cocamide TEA, ethoxilated linear alcohols, ethoxylatide alkyl phenols, fatty acid esters, amine, amide and urea derivatives, alkylpolyglugosides, ethyleneoxide/proyleneoxide copolymers, polyalcohols and ethoxylated polyalcohols, thiols and derivatives, fluorinated surfactants, silicon surfactant, Antifoamers, Defoamers, Detergents, Dispersants, Emulsifiers, Foaming agents, Foam boosters, Foam stabilizers, Solubilizers, Surfactant intermediates, Wetting agents, and the like.

In certain embodiments, the surfactant present in the composition is a siloxane surfactant. Of interest in certain embodiments are siloxane reactants that are substantially, if not completely, free of protic functionalities, e.g., hydroxy or amino functionalities. One class of such polysiloxane surfactants of interest is organo-functionally modified polysiloxanes of the general formula (III):

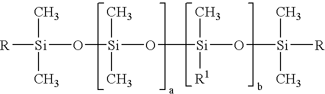

in which

R is in each case identical or different from R₁ or —CH₃,

R₁ is (CH₂)_{c}—O—(CH₂—CH(Ph)—O)ₑ—(CₙH₂ₙ₋ₓR²ₓ—O)_{d}—R³ and/or R¹′═CH₂—CHR*—Ph,

R* is H or —CH₃, R is an alkyl residue, for example having 1 to 5 carbon atoms, such as —CH₃,

Ph is a phenyl derivative having the general formula —(C₆H_{5-y}R⁴_{y})—

in which
R⁴ is a hydroxyl residue, an alkyl residue, e.g., having 1 to 6 carbon atoms or an alkoxy residue, e.g., having 1 to 6 carbon atoms, and
y is a number from 0 to 5, preferably 0 to 2,
R₃ is hydrogen, an alkyl chain, preferably having 1 and up to 18 carbon atoms, a benzyl residue, an alkyl-substituted benzyl residue preferably, having up to four carbon atoms in the alkyl residue, a group COR⁵ with a residue R⁵ which has an alkyl chain, e.g., having 1 to 18 carbon atoms, a group CONHR₆ with a residue R₆ which comprises a hydrogen atom or an alkyl chain, e.g., having 1 to 18 carbon atoms, or CO₂R⁷, which has an alkyl chain R⁷, e.g., having 1 to 18 carbon atoms,
c is from 2 to 6, such as 2 or 3,
d is from 3 to 70, such as from 3 to 50,
e is 0, ≧1, such as from 1 to 5,
with the proviso that if e is 0 the value of b is >1 and the residue R¹ is present at least once in the molecule, n is from 2 to 4, such as 2 or 3, x is 0 or 1, a is from 0 to 100, b is from 1 to 100, with the proviso that a+b=1 to 100, such as up to 50 and in particular up to 25.

The value of b and the value of a are to be understood as average values in the polymer molecule, since in certain embodiments the polysiloxanes of the invention are in the form of mixtures, which are generally equilibrated mixtures.

Such polysiloxane surfactants are further described in U.S. Pat. No. 7,018,458; the disclosure of which surfactants is herein incorporated by reference. Of interest in certain embodiments are the polysiloxane surfactants sold under the trademark TEGO®.

As with the viscosity modifiers, any surface tension modifier should be compatible with the chemistry in which it is to be used so that it does not adversely affect coupling yield (as defined above) by more than 5%. The amount of surface tension modifier, if present, in the composition may range from about 0.0001 to about 5% v/v, such as from about 0.001 to about 1% v/v and including from about 0.01 to about 0.1% v/v.

As indicated above, in certain embodiments the compositions include both a viscosity modifier and a surface tension modifier. In such embodiments, the amounts of each component in the solvent are chosen to provide for the desired physical properties of the composition, as described above. In such embodiments, the concentration of viscosity modifier may range from about 10⁻⁶ g/mL to about 10⁻¹ g/mL, such as from about 10⁻⁴ g/mL to about 10⁻² g/mL and including from about 10⁻³ g/mL to about 10⁻² g/mL. In such embodiments, the concentration of surface tension modifier may range from about 0.0001 to about 5% v/v, such as from about 0.001 to about 1% v/v and including from about 0.01 to about 0.1% v/v.

During use, the compositions further include at least a probe precursor, e.g., a nucleic acid precursor, such as a premade nucleic acid, e.g., and oligonucleotide, or a nucleic acid monomer, e.g., a nucleoside reagent of formula (1):

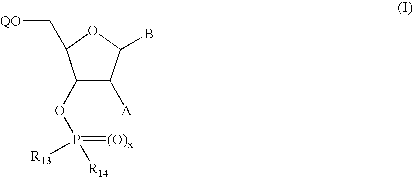

as described in greater detail above.

When present, concentration of nucleic acid precursor in the composition is one that is sufficient to provide for the desiring coupling during in situ synthesis. In certain embodiments, the concentration of precursor reagent ranges is about 0.01 g/mL or higher, such as about 0.1 g/mL or higher, and may range from about 0.01 g/mL to about 1% less than saturation in the given mixture, such as from about 0.1 g/mL to about 1 g/mL and including from about 0.2 g/mL to about 0.5 g/mL.

In certain embodiments, the compositions include an activator. As is known in the art, an activator is added to the solution to catalyze the formation of the internucleotide bond usually by the formation of a highly reactive intermediate. In phosphodiester, phosphotriester and H-phosphonate chemistry, Lewis Acid activators such as sulfonyl halides, sulfonyl azoles, pivaloyl halides, pivaloyl azoles, and adamatane carbonyl halides, are used to form mixed anhydrides that react to for the new internucleotide bond. In the case of phosphoramidite chemistry a protic acid catalyst is used to enhance the rate of displacement of the phosphorus-nitrogen bond. This rate can be additionally enhanced by using an azole catalyst that contains an acidic proton. Protic acid azole activators can include compounds such as, but not limited to, tetrazole, S-ethyl-thiotetrazole, 4-nitrotriazole, 5-benzylthio-tetrazole or dicyanoimidazole, although other acidic azoles can be used. An activator compound may be present in a concentration of about 0.05 molar up to about 1.0 molar. The concentration of these activators depends, at least in part, on the solubility of the azole in a solvent that supports phosphoramidite coupling.

In certain embodiments, the compositions may include both the precursor and the activator.

The subject compositions may be readily prepared using any convenient protocol. In making compositions of the invention, one may first select a solvent with a desired concentration of probe precursor and/or activator. Viscosity and surface tension of this composition may be measured and a viscosity modifier and/or surface tension modifier added as needed to alter the measured viscosity and surface tension to a figure indicated as suitable for intended use in a pulse-jet mediated in situ nucleic acid synthesis protocol. The solution of the desired viscosity and/or surface tension can then be tested in the pulse jet head to be used, both for reliability and uniformity of drop deposition. Reliability can be determined by repeatedly depositing drops from the same pulse jet over a long period of time (for example, 24 hours) and checking the number of drops which were in fact deposited. Uniformity can be determined by examining the drops deposited over time for size uniformity and for the presence of any deposited satellite drops, such as by capturing images of deposited drops with a linescan or other camera of suitable resolution and either manually examining the images or using image processing techniques to compare deposited drop sizes. If the results are unsatisfactory, viscosity and/or surface tension can be adjusted and the testing in the pulse jet head repeated. Such actual testing in the pulse jet head to be used reduces problems where the liquid being used is non-Newtonian (that is, the viscosity is substantially dependent upon the shear rate) and the viscosity measuring device is incapable of measuring viscosities at the shear rates in a typical piezo activated pulse jet. For example, a typical viscosity measuring device may be able to reach shear rates of only between 1-1800 sec⁻¹ whereas shear rates in a piezo activated pulse jet may be about 1 million sec⁻¹. Alternatively, a suitable initial viscosity and/or surface tension can just be estimated (for example, starting with a viscosity of 6 cps) and the testing in the pulse jet, viscosity and/or surface tension adjustment, and repeated testing in the pulse jet, performed as before. An upper and lower limit of suitable viscosity and/or surface tension for a particular pulse jet can be determined in this manner. Compatibility of the viscosity modifier and surface tension modifier with the chemistry may be tested by comparing the stepwise coupling yield of the nucleotide precursor in solution of the same liquid composition both with and without the presence of the viscosity modifier and surface tension modifier.

The above described compositions find use in a variety of different applications, including but not limited to: methods of in situ fabrication of nucleic acid arrays, particularly pulse-jet mediated fabrication of nucleic acid arrays, e.g., as described in great detail in the following section.

### Methods

As mentioned above, the compositions described above are particularly useful for fabricating an addressable polynucleotide array by in situ synthesis of polynucleotides on the array substrate. In one such embodiment, at each of the multiple different addresses on the substrate (e.g., at least one hundred, at least one thousand, or at least ten thousand addresses), the in situ synthesis cycle is repeated so as to form the addressable array with the same or different polynucleotide sequences at one or more different addresses on the substrate. In the array forming method, the compositions of the invention are deposited as droplets at those addresses using, for example, a pulse-jet printing system. The polynucleotides can be produced by disposing solutions (e.g., selected from four solutions, each containing a different nucleotide) on particular addressable positions in a specific order in an iterative process.

In array fabrication, different compositions of the nucleotide monomers and the activator can be deposited at different addresses on the substrate during any one cycle so that the different features of the completed array will have polynucleotides with different sequences. One or more intermediate further steps may be required in each cycle, such as the conventional oxidation, capping, and washing steps in the case of in situ fabrication of polynucleotide arrays (e.g., these steps can be performed by flooding the array surface with the appropriate reagents).

Embodiments of the methods lend themselves to synthesis of polynucleotides on array substrates in either the 3′-to-5′ or the 5′-to-3′ direction. In the former case, the initial step of the synthetic process involves attachment of an initial nucleotide to the array substrate at the 3′ position, leaving the 5′ position available for covalent binding of a subsequent monomer. In the latter case, the initial step of the synthetic process involves attachment of an initial nucleotide to the array substrate at the 5′ position, leaving the 3′ position available for covalent binding of a subsequent monomer. Following synthesis, the polynucleotide can, if desired, be cleaved from the solid support. The details of the synthesis in either the 3′-to-5′ or the 5′-to-3′ direction will be readily apparent to the skilled practitioner based on the prior art and the disclosure contained herein.

In one embodiment, a monomer nucleotide phosphoramidite is dissolved in the solvent including the organic salt, and the resulting solution is deposited upon the surface of the planar substrate, and the process is repeated multiple times, analogous to conventional polynucleotide synthesis, to form the target polynucleotide of interest.

The product array may contain any number of features, generally including at least tens of features, usually at least hundreds, more usually thousands, and as many as a hundred thousand or more features. All of the features may be different, or some or all could be the same. Each feature carries a predetermined moiety or a predetermined mixture of moieties, such as a particular polynucleotide sequence or a predetermined mixture of polynucleotides. The features of the array can be arranged in any desired pattern (e.g. organized rows and columns of features, for example, a grid of features across the substrate surface); a series of curvilinear rows across the substrate surface (for example, a series of concentric circles or semi-circles of features, and the like). In embodiments where very small feature sizes are desired, the density of features on the substrate can range from at least about ten features per square centimeter, or at least about 35 features per square centimeter, or at least about 100 features per-square centimeter, and up to about 1000 features per square centimeter, up to about 10,000 features per square centimeter, or up to 100,000 features per square centimeter. Each feature carries a predetermined nucleotide sequence (which includes the possibility of mixtures of nucleotide sequences).

In one embodiment, about 10 to 100 of such arrays can be fabricated on a single substrate (such as glass). In such embodiments, after the substrate has the polynucleotides on its surface, the substrate can be cut into substrate segments, each of which can carry one or two arrays. It will also be appreciated that there need not be any space separating arrays from one another. Where a pattern of arrays is desired, any of a variety of geometries can be constructed, including for example, organized rows and columns of arrays (for example, a grid of arrays, across the substrate surface), a series of curvilinear rows across the substrate surface (for example, a series of concentric circles or semi-circles of arrays), and the like.

The array substrate can take any of a variety of configurations ranging from simple to complex. Thus, the substrate could have generally planar form, as for example a slide or plate configuration, such as a rectangular or square or disc. In many embodiments, the substrate will be shaped generally as a rectangular solid, having a length in the range about 4 mm to 300 mm, usually about 4 mm to 150 mm, more usually about 4 mm to 125 mm; a width in the range about 4 mm to 300 mm, usually about 4 mm to 120 mm and more usually about 4 mm to 80 mm; and a thickness in the range about 0.01 mm to 5.0 mm, usually from about 0.1 mm to 2 mm and more usually from about 0.2 to 1 mm. The substrate surface onto which the polynucleotides are bound can be smooth or substantially planar, or have irregularities, such as depressions or elevations. The configuration of the array can be selected according to manufacturing, handling, and use considerations.

In array fabrication, the quantities of polynucleotide available are usually very small and expensive. Additionally, sample quantities available for testing are usually also very small and it is therefore desirable to simultaneously test the same sample against a large number of different probes on an array. Therefore, one embodiment of the invention provides for fabrication of arrays with large numbers of very small, closely spaced features. Arrays can be fabricated with features that can have widths (that is, diameter, for a round spot) in the range from a minimum of about 10 micrometers to a maximum of about 1.0 cm. In embodiments where very small spot sizes or feature sizes are desired, material can be deposited according to the invention in small spots whose width is in the range about 1.0 micrometer to 1.0 mm, usually about 5.0 micrometers to 0.5 mm, and more usually about 10 micrometers to 200 micrometers. Interfeature areas will typically (but not essentially) be present which do not carry any polynucleotide. It will be appreciated though, that the interfeature areas could be of various sizes and configurations.

Suitable substrates can have a variety of forms and compositions and can be derived from naturally occurring materials, naturally occurring materials that have been synthetically modified, or synthetic materials. Examples of suitable support materials include, but are not limited to, nitrocellulose, glasses, silicas, teflons, and metals (for example, gold, platinum, and the like). Suitable materials also include polymeric materials, including plastics (for example, polytetrafluoroethylene, polypropylene, polystyrene, polycarbonate, and blends thereof, and the like), polysaccharides such as agarose (e.g., that available commercially as Sepharose®, from Pharmacia) and dextran (e.g., those available commercially under the tradenames Sephadex® and Sephacyl®, also from Pharmacia), polyacrylamides, polystyrenes, polyvinyl alcohols, copolymers of hydroxyethyl methacrylate and methyl methacrylate, and the like.

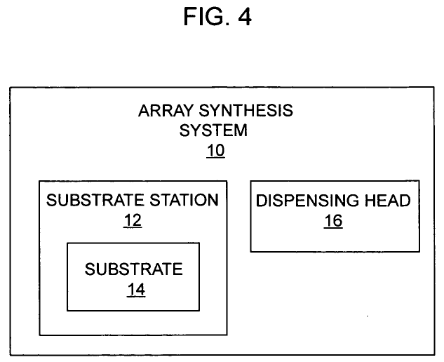

FIG. 4 (get from published application no. 20050287555) illustrates an embodiment of an array synthesis system 10 that uses organic salt solvents/co-solvents in the application to the deposition of nucleotide compounds to a suitable substrate (as described above), especially for the fabrication of polynucleotide arrays. The array synthesis system 10 depicted in FIG. 4 can be used to contact the insoluble planar substrate with the nucleotide composition of the invention, as described above. The array synthesis system 10 shown in FIG. 4 includes a substrate station 12 on which can be mounted a substrate 14. Substrate station 12 can include a vacuum chuck connected to a suitable vacuum source (not shown) to retain a substrate 14 without exerting too much pressure thereon, since substrate 14 is often made of glass. In addition, the array synthesis system 10 includes a dispensing head 16. The dispensing head 16 can be positioned to face the substrate station 12 by a positioning system. The positioning system includes a carriage connected to substrate station 12, a first transporter controlled by a processor, and a second transporter controlled by processor. The first transporter and carriage are used to execute one axis positioning of the substrate station 12 facing the dispensing head 16 by moving substrate station 12 in the x-axis direction, while the second transporter is used to provide y- and z-axis direction adjustment. Further, once substrate station 12 has been positioned facing dispensing head 12, the positioning system will be used to scan the dispensing head 12 across the mounted substrate 14, typically line by line (although other scanning configurations could be used).

The dispensing head 12 can be of a type commonly used in an ink jet type of printer and can, for example, have multiple drop dispensing orifices communicating with one or more chambers for holding either previously obtained solution including the organic salt as a solvent/co-solvent. Ejectors are positioned in the one or more chambers, each opposite a corresponding orifice. For example, each ejector can be in the form of an electrical resistor operating as a heating element under control of a processor (although piezoelectric elements could be used instead). Each orifice with its associated ejector and portion of the chamber, defines a corresponding pulse jet. In this manner, application of a single electric pulse to an ejector causes a droplet to be dispensed from a corresponding orifice. In particular, the dispensing head is an industrial inkjet print head.

Following contact of the substrate with the reagent composition of the invention for a period of time and under conditions sufficient for the nucleotide composition to react with the biomolecule on the substrate or with the substrate itself, as described above, the surface of the resultant array can be further processed as desired in order to prepare the array for use. For example, further iterations of the synthesis cycle can be performed for in situ synthesis. As another example, the array surface can be washed to removed unbound reagent (e.g. unreacted polymer, and the like). Any convenient wash solution and protocol can be employed (e.g. flowing an aqueous wash solution, e.g. water, methanol, acetonitrile, and the like) across the surface of the array, etc. The surface can also be dried and stored for subsequent use, etc.

Still other methods and apparatus for fabrication of polynucleotide arrays using solutions including organic salts are described in, e.g. U.S. Pat. No. 6,242,266 to Schleiffer et al., which describes a fluid dispensing head for dispensing droplets onto a substrate, and methods of positioning the head in relation to the substrate. U.S. Pat. No. 6,180,351 to Cattell and U.S. Pat. No. 6,171,797 to Perbost describe additional methods useful for fabricating polynucleotide arrays. Also of interest are the methods disclosed in published United States Patent Application Publication Nos. 20060078927; 20060057736; 20050287555; 20050244881; 20050233337; 20050214779; 20050214778; 20050214777; 20050019786; 20040219663; 20040203173; 20040185169; 20040166496; 20040152081; and 20030120035; the in situ fabrication methods disclosed in these various publications being specifically incorporated herein by reference.

Methods for fabrication of arrays can include, for example, using a pulse jet such as an inkjet type head to deposit a droplet of reagent solution for each feature. Such a technique has been described, for example, in PCT publications WO 95/25116 and WO 98/41531, and elsewhere. In such methods, the head has at least one jet which can dispense droplets of a fluid onto a substrate, the jet including a chamber with an orifice, and including an ejector which, when activated, causes a droplet to be ejected from the orifice. The head can be of a type commonly used in inkjet printers, in which a plurality of pulse jets (such as those with thermal or piezoelectric ejectors) are used, with their orifices on a common front surface of the head. The head is positioned with the orifice facing the substrate. Multiple fluid droplets (where the fluid comprises the nucleotide monomer, oligonucleotide, or polynucleotide dissolved in the solvent comprising an ionic liquid) are dispensed from the head orifice so as to form an array of droplets on the substrate (this formed array may or may not be the same as the final desired array since, for example, multiple heads can be used to form the final array and multiple passes of the head(s) may be required to complete the array).

The amount of fluid that is expelled in a single activation event of a pulse jet, can be controlled by changing one or more of a number of parameters, including the orifice diameter, the orifice length (thickness of the orifice member at the orifice), the size of the deposition chamber, and the size of the heating element, among others. The amount of fluid that is expelled during a single activation event is generally in the range about 0.1 to 1000 pL, usually about 0.5 to 500 pL and more usually about 1.0 to 250 pL. A typical velocity at which the fluid is expelled from the chamber is more than about 1 m/s, usually more than about 10 m/s, and can be as great as about 20 m/s or greater. As will be appreciated, if the orifice is in motion with respect to the receiving surface at the time an ejector is activated, the actual site of deposition of the material will not be the location that is at the moment of activation in a line-of-sight relation to the orifice, but will be a location that is predictable for the given distances and velocities.

It should be specifically understood, though, that the present disclosure is not limited to pulse jet type deposition systems. In particular, any type of array fabricating apparatus can be used to contact the substrate with the solution including the organic salt as a solvent/co-solvent, including those such as described in U.S. Pat. No. 5,807,522, or an apparatus that can employ photolithographic techniques for forming arrays of moieties, such as described in U.S. Pat. No. 5,143,854 and U.S. Pat. No. 5,405,783, or any other suitable apparatus which can be used for fabricating arrays of moieties. For example, robotic devices for precisely depositing aqueous volumes onto discrete locations of a support surface, i.e., arrayers, are also commercially available from a number of vendors, including: Genetic Microsystems; Cartesian Technologies; Beecher Instruments; Genomic Solutions; and BioRobotics. Other methods and apparatus are described in U.S. Pat. Nos. 4,877,745; 5,338,688; 5,474,796; 5,449,754; 5,658,802; and 5,700,637. Patents and patent applications describing arrays of biopolymeric compounds and methods for their fabrication include: U.S. Pat. Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,695; 5,624,711; 5,639,603; 5,658,734; WO 93/17126; WO 95/11995; WO 95/35505, WO 97/14706, WO 98/30575; EP 742 287; and EP 799 897. See also Beier et al. “Versatile derivatisation of solid support media for covalent bonding on DNA-microchips”, Nucleic Acids Research (1999) 27: 1970-1977.

### Array Synthesis Systems

Also provided are array synthesis systems, such as the system depicted in FIG. 4 and described above. Additional systems include those described in U.S. Pat. Nos. 6,180,351; 6,242,266; 6,306,599 and U.S. Pat. No. 6,420,180, the disclosure of the systems of each of these publications being specifically incorporated herein by reference. Aspects of the systems include at least one pulsejet head, if not all of the pulsejet heads, being loaded with, e.g., by being in fluid communication with a reservoir of, a composition of the invention, e.g., as described above.

### Kits

Also provided are kits of reagent compositions of the invention that find use, e.g., in methods of fabricating nucleic acid arrays. Kits of the invention may include a set of different compositions as used in a method of the present invention where each of the different compositions comprises a different reagent (for example, a different one of four nucleoside precursors, e.g., phosphoramidites) and/or activator. Other reagents of interest may also be included, e.g., wash fluids, buffers, etc.

The various components of the kit may be present in separate containers or certain compatible components may be precombined into a single container, as desired.

In addition to the above-mentioned components, the subject kits typically further include instructions for using the components of the kit to practice the subject methods (i.e., using the split-probe oligonucleotide in a method to evaluate the copy number and/or the methylation of a genomic region of interest). The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The following examples are offered by way of illustration and not by way of limitation.

### Experimental

Reagent formulations containing active reagents appropriate for the synthesis of DNA microarray using pule-jet technology to spatially control the step wise extension of the growing oligonucleotides were prepared according to Table 1, below. Corresponding formulations where the Polyethylene Glycol (PEG) addition had been omitted where also prepared for comparison. In all those formulations, the amount of active reagent has been chosen so as to efficiently enable the synthesis of oligonucleotides. The viscosities of those ink formulations were measured on a Brookfield DV-III rheometer using a CP-40 cup and reporting the viscosities at the maximum shear rate achievable. All measurements were performed at 25° C.

Table 2, below, shows the viscosity values with and without the added PEG (shaded). It can be readily observed that without added PEG, the viscosity of the different reagent formulations varies widely between 2.1 and 5.1 cP. After addition of various amounts of PEG polymer for different active reagents, it is apparent that not only the viscosity increased but also that the variation tightened to between 7.0 and 8.0 cP. This tightening of the physical properties of the ink formulations resulted in:

1) more uniform jetting performance (ejection of the droplets at more uniform speeds and with less satellites and less trajectory errors) between reagent formulation is for a determined set of jetting parameters (voltage, frequency, back-pressure);
2) more uniform printing performance (placement of droplets on solid support at better accuracy); and
3) ultimately better chemical synthesis of oligonucleotides (data not shown).

TABLE 1
Formulation of reagent formulation containing active
reagents for spatially controlled DNA synthesis.

| | Weight of | | |
|---|---|---|---|
| | phosphoramidite | Weight of PEG | Volume of PC |
| Active reagent | (g) | 35K (g) | (mL) |
| dA | 10 | 0.40 | 40 |
| dC | 10 | 0.40 | 40 |
| dG | 10 | 0.40 | 40 |
| dT | 10 | 0.75 | 50 |
| Tet | 10 | 2.04 | 68 |

PC is Propylene Carbonate and PEG 35K is polyethylene glycol polymer of average molecular weight 35,000 Da.

TABLE 2
Viscosity values in cP of ink formulations from Table 1. The values
in the shaded cells were obtained when PEG 35K was omitted.

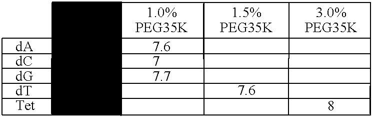

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A polar, aprotic composition comprising:
(a) a solvent; and
(b) at least one of:
(i) a viscosity modifier and
(ii) a surface tension modifier;
wherein said solvent, viscosity modifier and surface tension modifier are present in amounts chosen so that said composition is compatible for use in pulse-jet in situ nucleic acid synthesis protocols.

2. The composition according to claim 1, wherein said composition includes a viscosity modifier.

3. The composition according to claim 1, wherein said composition includes a surface tension modifier.

4. The composition according to claim 1, wherein said composition includes both a viscosity modifier and a surface tension modifier.

5. The composition according to claim 1, wherein said composition further comprises a nucleic acid precursor.

6. The composition according to claim 5, wherein said nucleic acid precursor is a phosphoramidite.

7. The composition according to claim 1, wherein said composition further comprises an activator.

8. The composition according to claim 1, wherein said viscosity modifier comprises a polyalkylene glycol.

9. The composition according to claim 8, wherein said polyalkylene glycol is a polyethylene glycol having a molecular weight from about 1 kDa to about 50 kDa.

10. The composition according to claim 8, wherein said viscosity modifier comprises two or more different polyethylene glycols of different molecular weights.

11. The composition according to claim 1, wherein said surface tension modifier is a surfactant.

12. The composition according to claim 11, wherein said surfactant is a siloxane surfactant.

13. The composition according to claim 1, wherein said solvent is a polar, aprotic, high boiling solvent.

14. The composition according to claim 1, wherein said composition has a boiling point of about 120 or higher.

15. A method comprising:
contacting a surface of a solid support with a polar, aprotic composition comprising:
(a) a solvent;
(b) at least one of:
(i) a viscosity modifier and
(ii) a surface tension modifier; and
(c) at least one of:
(i) a nucleic acid precursor; and
(ii) an activator;
wherein said solvent, viscosity modifier and surface tension modifier are present in amounts chosen so that said composition is compatible for use in pulse-jet in situ nucleic acid synthesis protocols.

16. The method according to claim 15, wherein said composition comprises a nucleic acid precursor.

17. The method according to claim 15, wherein said composition comprises an activator.

18. The method according to claim 15, wherein said method is a method of covalently bonding a nucleic acid precursor to a functional group present on said surface.

19. The method according to claim 18, wherein said method is a method of fabricating a nucleic acid array.

20. A method of fabricating an addressable array of nucleic acids bound to a surface of a solid support, said method comprising:
(a) depositing drops which contain nucleic acid precursors onto the said surface so that said nucleic acid precursors bind to said surface through a linker, wherein said drops comprise said nucleic acids precursors in a composition comprising a solvent and at least one of a viscosity modifier and a surface tension modifier; and
(b) repeating (a) multiple times wherein a nucleic acid precursor deposited in a prior cycle becomes the linker for a nucleic precursor deposited in a subsequent cycle, so as to fabrication said array of nucleic acids bound to a surface of a solid support.

21. The method according to claim 20, wherein said nucleic acid precursors are phosphoramidites.

22. An apparatus for synthesizing an array of biopolymers on the surface of a support, said apparatus comprising one or more pulse jet heads for dispensing a fluid formulation, wherein at least one of said pulse jet heads is loaded with a fluid formulation comprising:
(a) a solvent;
(b) at least one of:
(i) a viscosity modifier and
(ii) a surface tension modifier; and
(c) at least one of:
(i) a nucleic acid precursor; and
(ii) an activator;
wherein said solvent, viscosity modifier and surface tension modifier are present in amounts chosen so that said composition is compatible for use in pulse-jet in situ nucleic acid synthesis protocols.

23. A kit comprising a set of two or more different fluid compositions, wherein each fluid composition comprises:
(a) a solvent;
(b) at least one of:
(i) a viscosity modifier and
(ii) a surface tension modifier; and
(c) at least one of:
(i) a nucleic acid precursor; and
(ii) an activator;
wherein said solvent, viscosity modifier and surface tension modifier are present in amounts chosen so that said composition is compatible for use in pulse-jet in situ nucleic acid synthesis protocols.
